(19) United States
(12) Patent Application Publication
(10) Pub. No.: US 2011/0053274 A1
(43) Pub. Date: Mar. 3, 2011

(54) LAC EXPRESSION SYSTEM

(75) Inventors: Frederick Blattner, Madison, WI (US); Dimitry Schevchenko, Madison, WI (US); David Frisch, Fitchburg, WI (US); John Campbell, Oak Park, IL (US)

(73) Assignee: SCARAB GENOMICS LLC, Madison, WI (US)

(21) Appl. No.: 12/745,443

(22) PCT Filed: Nov. 26, 2008
(86) PCT No.: PCT/US08/84964

§ 371 Date: Oct. 26, 2010

Related U.S. Application Data
(60) Provisional application No. 60/991,608, filed on Nov. 30, 2007.

(51) Int. Cl. C12N 15/87; C07H 21/04; C12N 1/21

(52) U.S. Cl. 435/471; 536/24.1; 435/252.33

(57) ABSTRACT

Provided herein is a nucleic acid comprising a mutant lac operator operably linked to a gene of interest, a host cell comprising the nucleic acid, and a method of using the host cell to express the gene of interest. Also provided is a recA-mediated cloning method.

### FIELD OF THE INVENTION

This invention relates to a method of expressing a gene of interest that is regulated by a mutant lac operator with increased affinity for LacI repressor protein, to a nucleic acid containing the mutant lac operator, and to a host cell containing the nucleic acid. The invention also relates to a method of cloning nucleic acids using recA-mediated recombination.

### BACKGROUND OF THE INVENTION

Manufacturing proteins and enzymes for pharmaceutical and industrial applications via heterologous gene expression is an efficient and economical means of production. The gram-negative bacterium, Escherichia coli (E. coli) remains the most commonly-used host cell platform for expression genes of interest in both research and industry. Expressing heterologous proteins is often sensitive to fermentation and cell growth parameters and frequently, the optimal induction regimen for one protein is different from that of another protein, thus requiring a flexible method of induction that can be varied to maximize the yield of a given product. Gene products that are toxic to the host expression system are particularly troublesome, and many means for tightly regulating gene expression are cumbersome and ill-suited for high-throughput, high-volume applications.

Strategies for expressing genes often include placing a gene of interest under the control of a promoter that is regulated by an RNA polymerase, which is often encoded by a gene controlled by an inducible promoter. One such expression system is the bacteriophage T7 promoter controlling expression of a gene of interest and the RNA polymerase T7 gene 1 under control of the lac promoter, as described in U.S. Pat. Nos. 6,569,669, 5,869,320, 5,693,489, and 4,952,496, the contents of which are incorporated herein by reference. Other expression systems include a rhamnose-inducible T7 polymerase gene and an arabinose-inducible gene, as described in U.S. patent application Ser. No. 10/537,075 and U.S. Pat. No. 5,028,530, respectively, the contents of which are incorporated herein by reference.

Current gene expression systems are hampered by a number of factors, including: basal expression levels of the RNA polymerase in the absence of the inducing agent (i.e., “leaky” promoters); rare and expensive inducing agents (e.g., iso-propyl beta-thiogalactosidase [IPTG]); limited transport of the inducing agent into the host system; sluggish kinetic response of the RNA polymerase expression upon introduction of the inducing agent to the host system; and, loss of regulation in operator-repressor systems leading to undesirable expression of the RNA polymerase even in the absence of the inducing agent. It is thus desirable to develop alternative expression systems.

### SUMMARY OF THE INVENTION

Provided herein is a nucleic acid comprising a mutant lac operator that is operably linked to a gene of interest. The mutant lac operator may have increased affinity for a LacI repressor protein. The nucleic acid may be a plasmid or chromosome. The affinity of the lac operator for the LacI repressor protein may be increased by at least 5-fold. The sequence of the mutant lac operator may comprise any one of SEQ ID NOs: 2-6. The gene of interest may encode a protein with biological activity such as an antibody or enzyme. The protein may be T7 gene 1 or trfA.

Also provided is a host cell comprising the nucleic acid. The host cell may comprise a lacI gene, which may be mutant. The lacI allele may encode a mutant LacI repressor protein that may bind a lac operator with increased affinity. The host cell may also comprise an inactive lacZ gene, which may prevent the host cell from cleaving lactose into glucose and galactose, or converting lactose to allolactose.

Further provided is a method of expressing a gene of interest by contacting the host cell with a LacI allosteric effector. The allosteric effector may lead to expression of the gene of interest. The LacI allosteric effector may be derived from lactose, and may be a lactose analog such as isopropyl-β-D-thiogalactopyranoside.

Also provided is a method of cloning a first nucleic acid using a host cell comprising recA. Within the host cell, a first nucleic acid, which may be circular, may be recombined with a second nucleic acid by contacting the nucleic acids in the host cell. The recA may be located in the host cell genome or on a plasmid. The first nucleic acid may be a plasmid, and the second nucleic acid may be a host cell chromosome. The first nucleic acid may comprise at least two regions of sequence identity to regions on the second nucleic acid. The first nucleic acid may also comprise a selectable marker that conveys kanamycin resistance or encodes green fluorescent protein. After the nucleic acids recombine, the recA plasmid may be removed. The host cell may be a gram-negative bacterium such as E. coli.

### BRIEF DESCRIPTION OF THE DRAWINGS

FIG. 1 is a schematic representation of a construction strategy for a lac-T7 polymerase replacement via initial (inter-chromosomal) recombination.

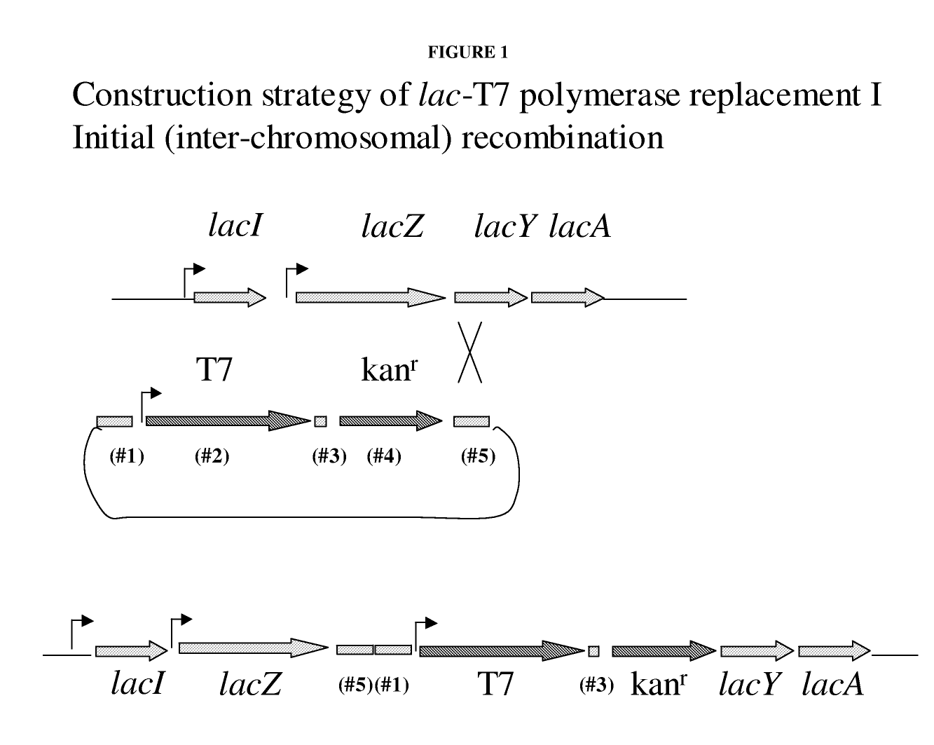

FIG. 2 is a schematic representation of a construction strategy for a lac-T7 polymerase replacement via secondary (intra-chromosomal) recombination.

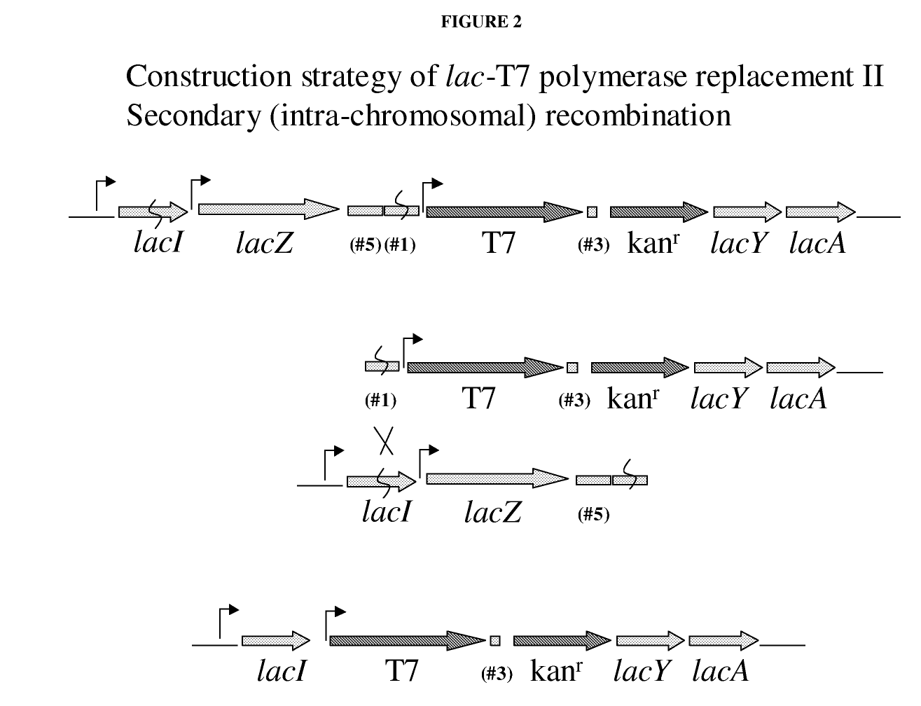

FIG. 3 shows the primers and templates used in the first round of PCR in generating a lac super operator T7 polymerase construct.

FIG. 4 shows the primers and templates used in a the second and third round of PCR in generating a lac super operator T7 polymerase and construct.

FIG. 5 shows a schematic of a lac super operator T7 polymerase construct and the primers used to generate it.

FIG. 6 depicts a strategy for cloning and identifying a lac super operator T7 polymerase and the photograph of a gel resulting from cloning the construct.

FIG. 7 depicts a strategy and primers used for confirming the recombination of a lac super operator T7 polymerase construct into an E. coli genome, as well as a photograph of a gel resulting from the confirmation strategy.

FIG. 8 depicts a strategy and primers used, and a photograph of a gel resulting from a screen to confirm intramolecular RecA-mediated recombination event between the 5′ portion of lacY and the E. coli chromosomal copy of lacY, which resulted in “collapse” of the region of the E. coli chromosome in which the endogenous lacZ resided.

FIG. 9 shows the primers and templates used to generate a lacZ back construct.

FIG. 10 shows a schematic of a lacZ back construct and the primers used to generate it.

FIG. 11 depicts a strategy for introducing lacZ into a lac super operator T7 polymerase E. coli strain and a photograph of a gel resulting from the strategy.

FIG. 12 depicts a strategy and primers used to confirm the introduction of lacZ into a lac super operator T7 polymerase E. coli strain, and a photograph of a gel resulting from the strategy.

FIG. 13 depicts a strategy and primers for confirming the generation of an E. coli strain that contains a lac super operator T7 polymerase and lacZ, but lacks kanamycin resistance, and a photograph of a gel resulting from the strategy.

FIG. 14 shows a schematic of a lac super operator T7 strain and the primers used to construct it.

FIG. 15 shows a schematic of a suicide vector comprising oriV which allows the suicide vector to replicate in the presence of TrfA protein, a green fluorescent protein-kanamycin resistance marker, and a multiple cloning site, which can be used in a nucleic acid recombination strategy.

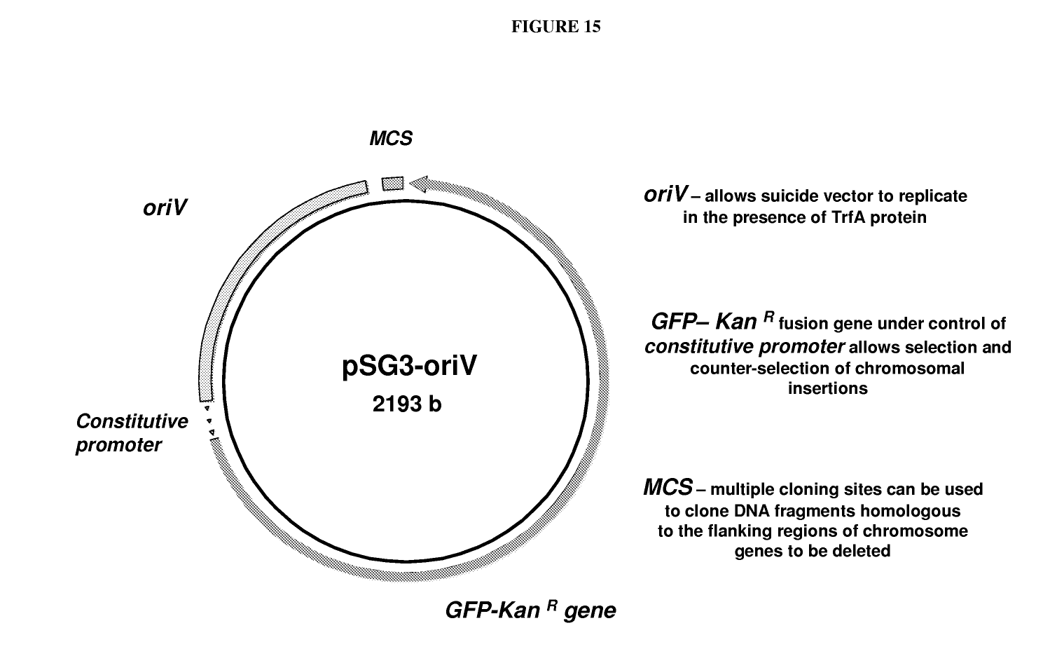

FIG. 16 shows a schematic of a possible first step of a nucleic acid recombination strategy.

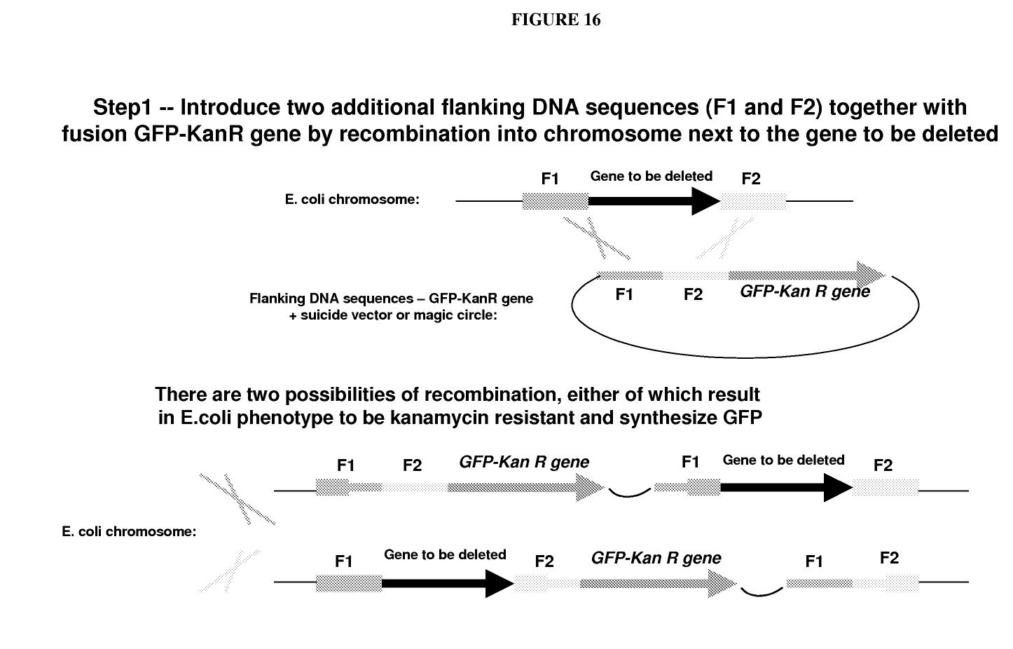

FIG. 17 shows a schematic of a possible second step of a nucleic acid recombination strategy.

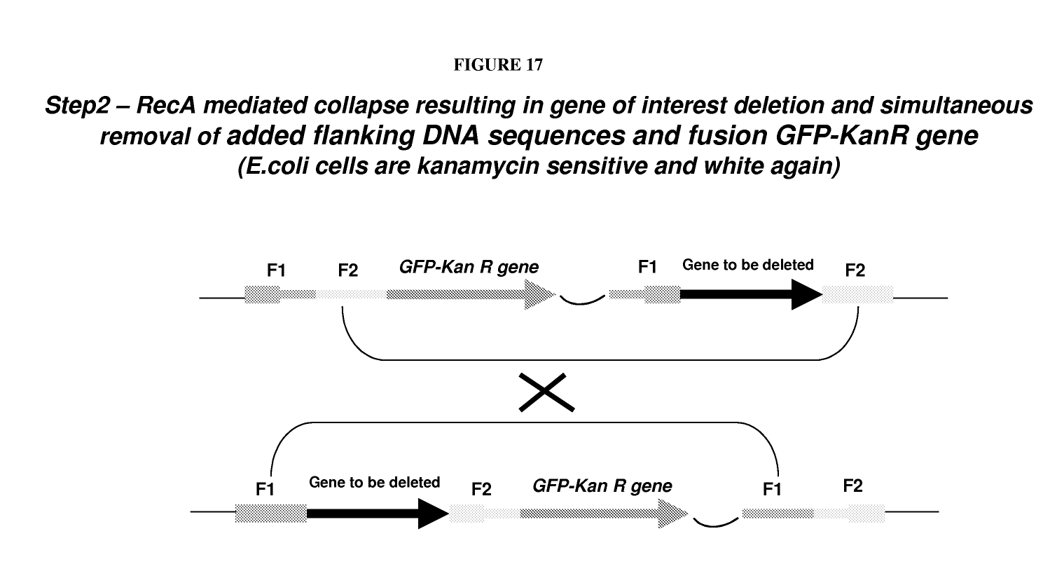

FIG. 18 is a schematic representation of an alternative construction strategy for a lac-T7 polymerase replacement via initial (inter-chromosomal) recombination.

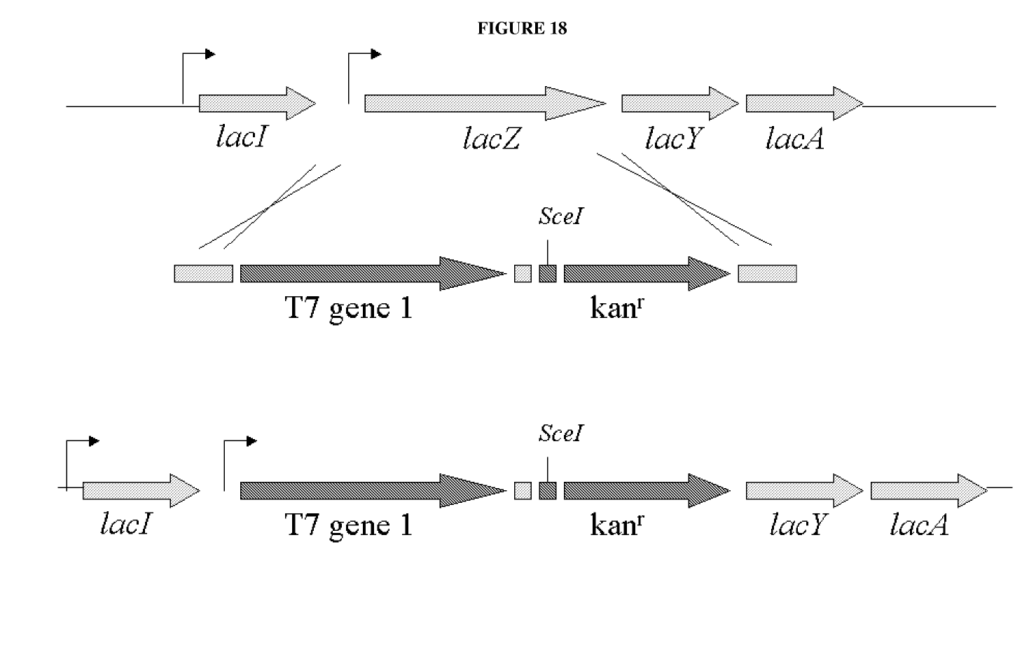

FIG. 19 is a schematic representation of an alternative construction strategy for a lac-T7 polymerase replacement via secondary (intra-chromosomal) recombination.

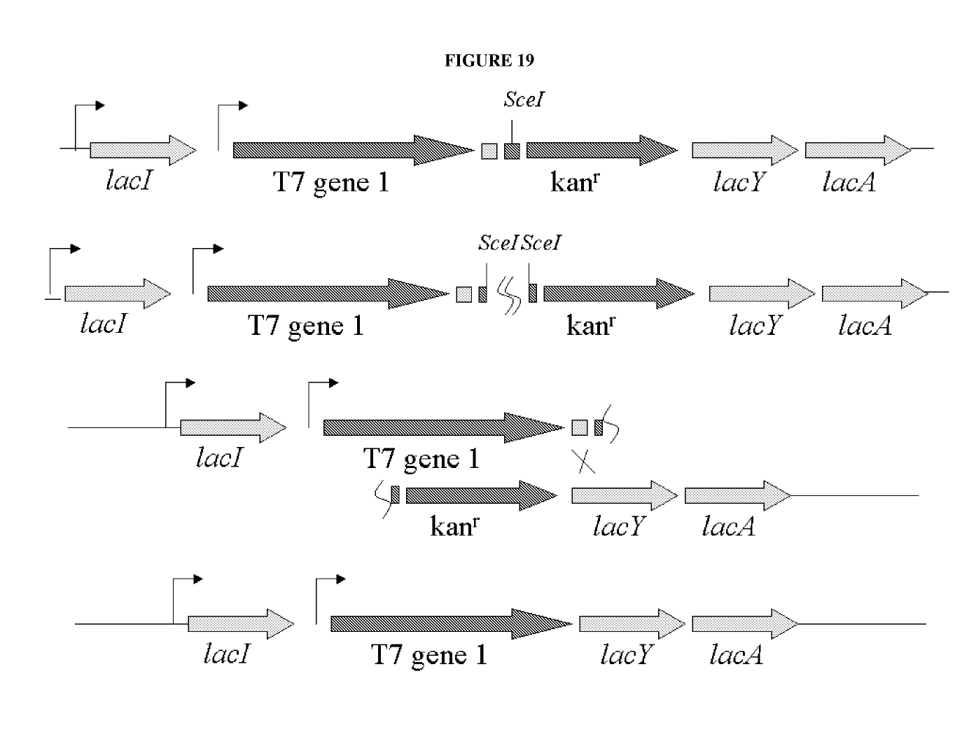

FIG. 20 shows the primers and templates used in an alternative first round of PCR in generating a lac super operator T7 polymerase construct.

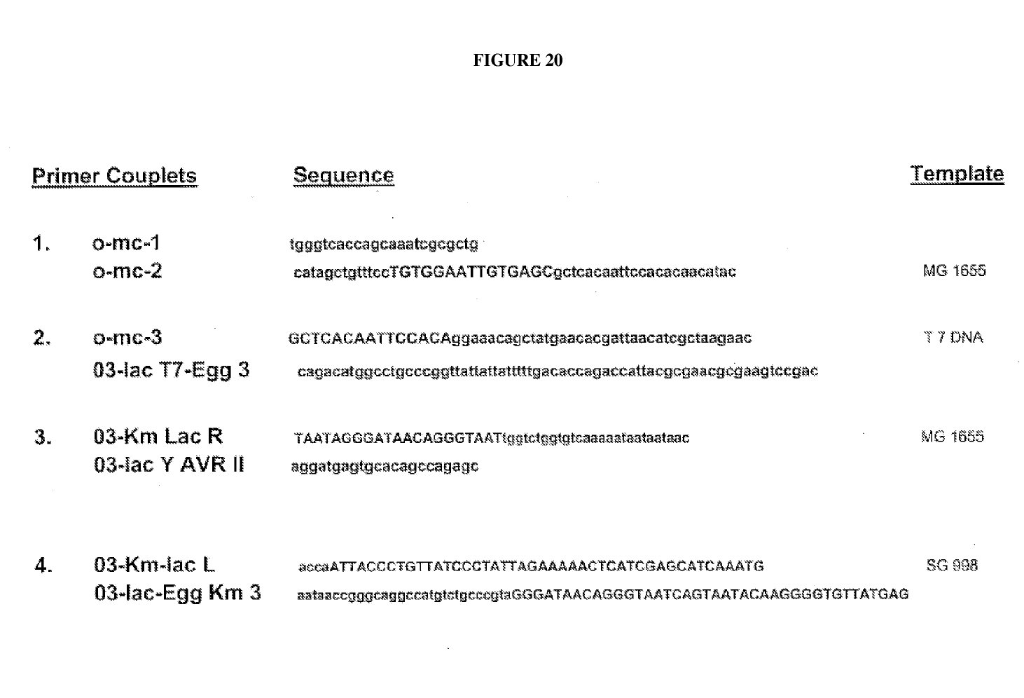

FIG. 21 shows the primers and templates used in an alternative second and third round of PCR in generating a lac super operator T7 polymerase and construct.

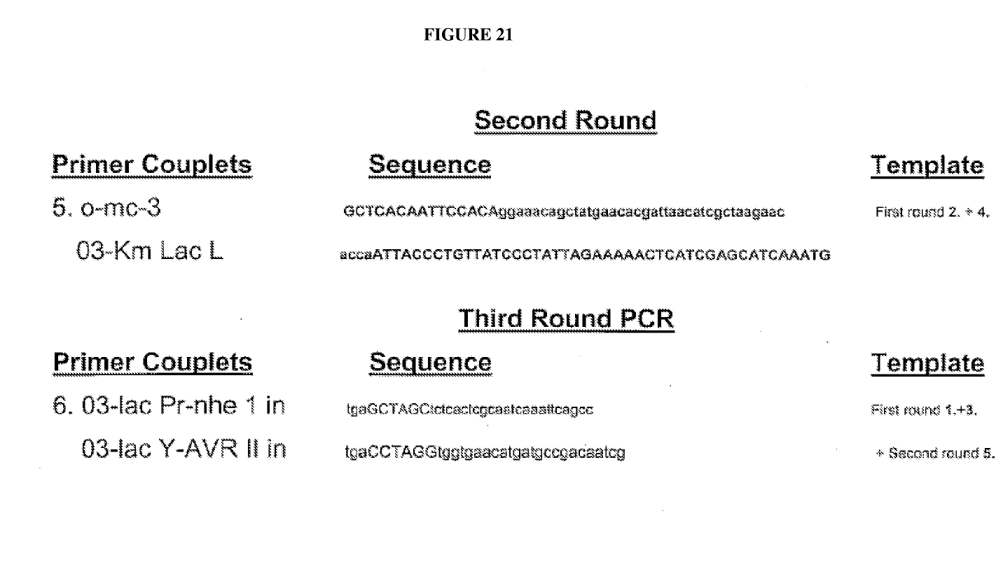

FIG. 22 shows an alternative schematic of a lac super operator T7 polymerase construct and the primers used to generate it.

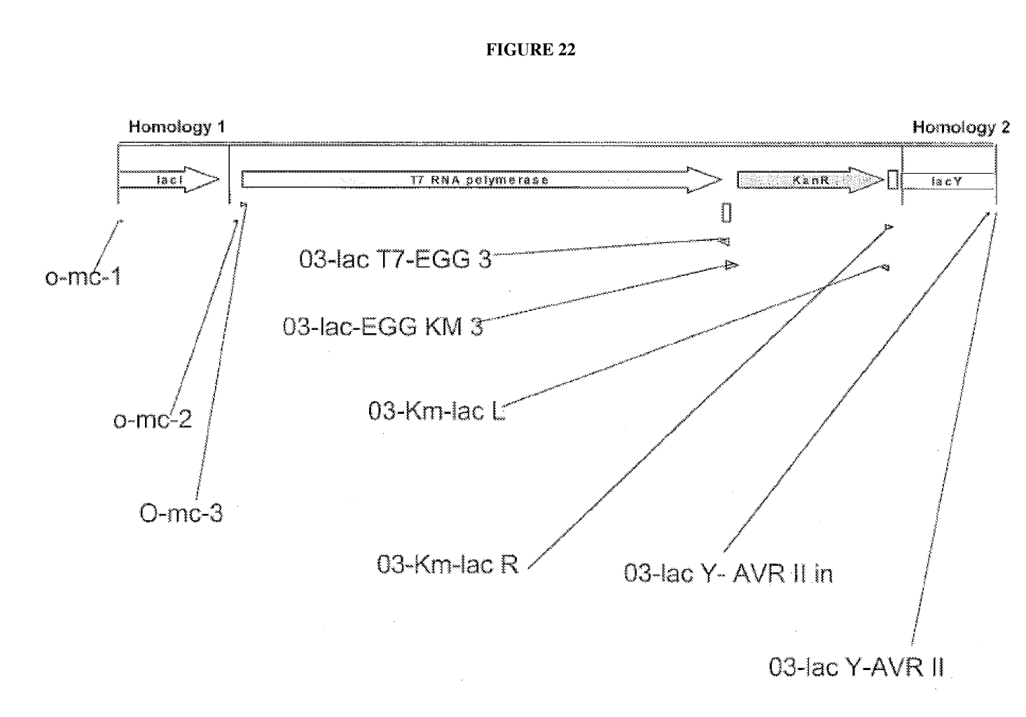

### DETAILED DESCRIPTION

The lac operon comprises a regulatory domain, the lac operator, and three genes involved in lactose uptake and catabolism, lacZ, lacY, and lacA (reviewed in Vilar et al, 2003, J Cell Biol, 161(3):471-476). The lac operator is regulated by the LacI repressor protein, which belongs to the helix-turn-helix family of transcriptional regulators. The LacI repressor protein functions as a homo-tetramer capable of binding any of three sites present in the lac operator with varying affinity. In the absence of lactose, the LacI repressor protein is capable of mediating more than a 1000-fold repression of the lac operator, which occurs predominantly via stearic hindrance between the LacI repressor and RNA polymerase caused by an interaction between the LacI repressor protein and a nucleic acid sequence close to the transcriptional start site of the lac operon (Besse et al, 1986, EMBO J, 5(6):1377 81; Lehming et al, 1987, EMBO J, 6(10):3145 3153). Lactose derivatives are capable of binding to the LacI repressor protein and inducing a conformational shift in the protein, which reduces the affinity of the LacI repressor protein for the lac operator and allows increased expression of the lac operon.

Provided herein are methods for expressing a gene of interest using components of the lac system. In the absence of an inducing agent, basal levels of the gene of interest can be reduced and more tightly-controlled by increasing occupancy time, and hence, repression, of the lac operator by the LacI repressor protein. Previously, naturally-isolated variant promoters have been used in this application. These include lacI^{q}, which is localized to the promoter of the gene encoding the LacI repressor protein. The lacI^{q} mutation causes elevated levels of the LacI repressor protein and therefore increased repression of the lac operon. Operator sequence variants capable of forming very tight complexes with LacI have been identified by systematic analysis of LacI binding to randomized oligonucleotides in in vitro studies (Lehming et al; Sadler et al, 1983, Proc Natl Acad Sci USA 80:6785-89), but most have not been tested for in vivo applications. The methods provided herein take advantage of variant lac operators with increased affinity for LacI repressor protein, obviating the need for increased levels of LacI repressor protein and simplifying purification of a protein produced by a gene of interest.

### 1. DEFINITIONS

The terminology used herein is for the purpose of describing particular embodiments only and is not intended to be limiting. As used in the specification and the appended claims, the singular forms “a,” “an” and “the” include plural referents unless the context clearly dictates otherwise.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6,9, and 7.0 are explicitly contemplated.

a. Chromosome

“Chromosome” used herein may be a nucleic acid packaged in a cell. The nucleic acid may be a single piece of DNA. The DNA may be linear or circular. The DNA may comprise 1, 10, 100, 1000, 2000, 3000, 4000, 5000, or 10000 genes. The DNA may also comprise regulatory elements and/or intervening nucleotide sequences. The DNA may also comprise an origin of replication. The origin of replication may be an oriC. The DNA may be natural to the cell. The DNA may also be exogenous to the cell. For example, the DNA may be an artificial chromosome. The DNA may also be compacted, such as in a cytologically visible nucleoid.

b. Gene

“Gene” used herein may be a natural (e.g., genomic) or synthetic gene comprising transcriptional and/or translational regulatory sequences and/or a coding region and/or non-translated sequences (e.g., introns, 5′- and 3′-untranslated sequences). The coding region of a gene may be a nucleotide sequence coding for an amino acid sequence or a functional RNA, such as tRNA, rRNA, catalytic RNA, siRNA, miRNA or antisense RNA. A gene may also be an mRNA or cDNA corresponding to the coding regions (e.g., exons and miRNA) optionally comprising 5′- or 3′-untranslated sequences linked thereto. A gene may also be an amplified nucleic acid molecule produced in vitro comprising all or a part of the coding region and/or 5′- or 3′-untranslated sequences linked thereto.

c. Host Cell

“Host cell” used herein may be a naturally occurring cell or a transformed cell that may contain a vector and may support replication of the vector. Host cells may be cultured cells, explants, cells in vivo, and the like. The host cell may be a prokaryotic cell such as E. coli, Salmonella species, Haemophilus influenzae, Lactococcus lactis, and Shigella species. The host cell may also be a eukaryotic cell such as yeast, insect, and amphibian, or a mammalian cell such as CHO and HeLa.

d. Inactive

“Inactive” used herein may mean an inactive gene. The inactive gene may comprise a mutation. The mutation may cause loss-of-function of the gene. The inactive gene may also comprise a deleted sequence compared to the wild-type sequence. The deleted sequence may comprise a portion of the gene. The deleted sequence may also comprise the entirety of the gene. The inactive gene may also comprise a transposon. The transposon may be located 5′ or 3′ of the gene. The transposon may also be located within the gene. The inactive gene may insure that the host cell is incapable of producing a protein encoded by the gene.

e. Insert Site

“Insert site” used herein may mean a nucleic acid with a sequence comprising a restriction site or recombinant site, which upon digestion with a restriction enzyme may allow a second nucleic acid nucleic acid to be inserted. The insert site may comprise a multiple cloning site.

f. Mutant

“Mutant” or “mutation” used herein may mean a nucleic acid or polypeptide comprising one or more substitutions, deletions, or insertions compared to a referenced nucleic acid or polypeptide.

g. Nucleic Acid

“Nucleic acid” or “oligonucleotide” or “polynucleotide” used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

h. Operably Linked

“Operably linked” used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

i. Peptide

A “peptide” or “polypeptide” is a linked sequence of amino acids and may be natural, synthetic, or a modification or combination of natural and synthetic.

j. Plasmid

“Plasmid” as used herein may mean a nucleic acid, wherein the nucleic acid has a circular structure. The plasmid may be extrachromosomal. The plasmid may have a length of 100; 1000; 2000; 5000; 10,000; 20,000; 30,000; 40,000; 50,000; 60,000; 70,000; 80,000; 90,000; 100,000; 110,000; 120,000; 130,000; 140,000; 150,000; 160,000; 170,000; 180,000; 190,000; 200,000; 210,000; 220,000; 230,000; 240,000; 250,000; 260,000; 270,000; 280,000; 290,000; 300,000; 310,000; 320,000; 330,000; 340,000; 350,000; 360,000; 370,000; 380,000; 390,000; or 400,000 nucleotides. The plasmid may be a low-, medium-, or high-copy plasmid. The plasmid may comprise an origin of replication. The plasmid may also comprise a selectable marker. The plasmid may also comprise a screening marker. The plasmid may also include a multiple cloning site, wherein the multiple cloning site comprises restriction sites. The restriction sites may be used for cloning an additional nucleic acid. The plasmid may be pTYB 1, pTYB2, pTYB 11, pTYB12, pLitmus29, pMAL-C2X, pMAL-C2T, pMALp2, pMALc2, pMALcR1, pET3, pET3a, pET11a, pET11d, pET15b, pET17, pET21d(+), pET22b, pET28a, pET29a, pET30a, pET 42b(+), pET 42b(+), pET44b(+), pET44b(+), pKK233-2, pKK22-33, pRSETA, pRSETB, pRSETC, pTP2P, pTRC99A, pGEX2T, pGEX3X, pGEX-2TK, pAT153, HAT4, pPROEXHTb, pTZ18, pTZ19, PTZ19R^{J}L1, PTZ19R^{J}L2, pACYC177, pACYC184A, pGEM1, pGEM4Z, pGEM 7Zf(+), pLitmus29, pGEMEX-1, pcDNA3, pCR-Script (sk+), pBluescript II SK(+), pBluescript II SK(−), pBluescript II KS(+), pBluescript II KS(−), pSELECT-1, pCR-Blunt-II[-TOPO], pLysS, pBR322, pUC18, pUC19, pUC118, pUC120, pUC121, pUC-4K, or variants thereof.

k. Promoter

“Promoter” or “operator” as used herein may mean a synthetic or naturally-derived molecule which is capable of conferring, activating or enhancing expression of a nucleic acid in a cell. A promoter may comprise one or more specific transcriptional regulatory sequences to further enhance expression or to alter the spatial expression or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

l. Screening Marker

“Screening marker” used herein may mean any gene which confers a phenotype on a host cell in which it is expressed to facilitate the screening or detection of cells which are transfected or transformed with a genetic construct. Representative examples of screening markers include the beta-galactosidase (β-gal)-encoding gene, beta-glucuronidase (GUS) gene, chloramphenicol acetyltransferase (CAT) gene, green fluorescent protein (GFP)-encoding gene, yellow fluorescent protein (YFP)-encoding gene, and luciferase gene.

m. Selectable Marker

“Selectable marker” as used herein may mean any gene that confers a phenotype on a host cell in which it is expressed to facilitate the identification or selection of cells that are transfected or transformed with a genetic construct. Representative examples of selectable markers include the ampicillin-resistance gene (Amp^{r}), tetracycline-resistance gene (Tc^{r}), bacterial kanamycin-resistance gene (Kan^{r}), zeocin resistance gene, the AURI-C gene which confers resistance to the antibiotic aureobasidin A, phosphinothricin-resistance gene, neomycin phosphotransferase gene (nptII), hygromycin-resistance gene, and green fluorescent protein (GFP). Selection may be performed by contacting a host cell comprising the selectable marker with a selection agent such as an antibiotic.

n. Substantially Complementary

“Substantially complementary” as used herein may mean that a first sequence is at least 60% to 99% identical to the complement of a second sequence over a region of 8 to 100 or more nucleotides, or that the two sequences hybridize under stringent hybridization conditions.

o. Substantially Identical

“Substantially identical” as used herein may mean that a first and second sequence are at least 50% to 99% identical over a region such as 2 to 100 or more nucleotides or amino acids, or with respect to nucleic acids, if the first sequence is substantially complementary to the complement of the second sequence.

p. Symmetrical

“Symmetrical” or “substantially symmetrical” as used herein to refer to a nucleic acid, may mean a nucleic acid comprising a sequence, wherein the first half of the sequence is substantially complementary to the second half thereof. A symmetrical sequence may be perfectly symmetrical, which may mean that the first half of the sequence is completely complementary to the second half thereof. The symmetrical sequence may have a center of symmetry, which center may mean a position between two nucleotides, wherein the position is precisely between the first half and second half of the symmetrical sequence.

q. Variant “Variant” as used herein to refer to a peptide or polypeptide, may mean a peptide or polypeptide that differs in amino acid sequence by the insertion, deletion, or conservative substitution of amino acids, but retain at least one biological activity. For purposes of this invention, “biological activity” includes, but is not limited to, the ability to be bound by a specific antibody. The variant may be a portion of a referenced protein sequence or a protein that is substantially identical to a referenced protein. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated herein by reference. Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Variant” as used herein to refer to a nucleic acid may mean (i) a portion of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

r. Vector

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome. The vector may comprise a selectable marker or a screening marker.

### 2. NUCLEIC ACID

A mutant lac operator that has an increased affinity for LacI repressor protein may be used to express a gene of interest. Provided herein is a nucleic acid, comprising a mutant lac operator operably linked to an insert site. The nucleic acid may be a vector, such as a plasmid. The nucleic acid may also be a chromosome.

The mutant lac operator may be capable of binding to a LacI repressor protein. The mutant lac operator may comprise a mutation that causes the mutant lac operator to have increased affinity for a LacI repressor protein compared to a wild-type lac operator, the sequence of which wild-type lac operator may comprise SEQ ID NO: 17. The sequence of the wild-type lac operator may also comprise SEQ ID NO: 1. The affinity of the mutant lac operator for a LacI repressor protein may be at least 2- to 20-fold higher than the affinity of a wild-type lac operator for the LacI repressor protein. The increased affinity may result in greater repression of the mutant lac operator by LacI repressor protein compared to a wild-type operator.

The mutant lac operator may comprise a sequence with an increased degree of symmetry compared to the sequence of a wild-type lac operator. The mutant lac operator may comprise a substantially symmetrical or perfectly symmetrical sequence. The symmetrical sequence may comprise at least 18 to 50 nucleotides. The center of symmetry may be 9 to 17 base pairs downstream from the start of transcription at the lac operator. The sequence of the mutant lac operator may also comprise the sequence 5′-TGTGGAATTGTGAGCGCTCACAATTC CACA-3′ (SEQ ID NO: 18). The sequence of the mutant lac operator may also comprise any one of SEQ ID NOS: 2-8.

The insert site may allow introduction of a gene of interest. For example, the insert site may comprise restriction sites (e.g., a multiple cloning site). Alternatively, the insert site may comprise a site for recombination.

The gene of interest may be trfA or T7 gene 1. The gene of interest may encode a protein with biological activity. The protein may be an antibody, an enzyme, a hormone, or a structural protein. The protein may also be interferon-α2b, alglucerase, imiglucerase, human insulin, interferon-β1a, somatropin, epoetin alpha, erythropoetin, clotting factor VIII, sermorelin, trastuzumab, palivizumab, alteplase, human growth hormone, or human albumin.

### 3. HOST CELL

Provided herein are host cells that may be used to express a gene of interest. The host cell may be a prokaryote. The prokaryote may be a bacterium. The bacterium may be E. coli.

The host cell may comprise a nucleic acid as described herein. The insert site of the nucleic acid may replace the lacZ gene of the host cell. The insert may allow introduction of a gene of interest under control of a LacI repressor protein while also reducing or eliminating metabolism of lactose by a host cell.

The host cell may also comprise a lacI gene. Methods of introducing the nucleic acid to the host on a vector or in the chromosome are well known in the art, for example, as described in Ausubel (ed.) et al (2006, Current Protocols in Molecular Biology, John Wiley & Sons, Inc.), the contents of which are incorporated herein by reference.

The lacI gene may encode a LacI repressor protein. The lacI gene may be a mutant lacI allele. The mutant lacI allele may encode a mutant LacI repressor protein that is capable of binding a lac operator with increased affinity compared to a wild-type LacI repressor protein. The affinity of the mutant LacI repressor protein for a lac operator may be at least 2-, 3-, 4-, 5-, 6-, 7-, 8-, 9-, 10-, 20-, 30-, 40-, 50-, 60-, 70-, 80-, 90-, 100-, 200-, 300-, 400-, 500-, or 1000-fold higher than the affinity of a wild-type LacI repressor protein for the lac operator. The sequence of the LacI repressor protein may comprise SEQ ID NO: 9 or 16. The LacI repressor may also comprise a recognition helix with a sequence consisting of SEQ ID NO: 10. The sequence of the recognition helix may also consist of any one of SEQ ID NOS: 11-16.

In the absence of β-galactosidase activity, lactose is not metabolized and hence cannot efficiently be used to induce a LacI repressor protein. The host cell may comprise an inactive lacZ gene. The lacZ gene may encode β-galactosidase. The inactive lacZ gene may prevent the host cell from cleaving lactose into glucose and galactose or converting lactose to allolactose.

### 4. METHOD OF EXPRESSION

Provided herein are methods of expressing a gene of interest. A method of expressing a gene of interest may comprise a host cell as described herein. The gene of interest may be expressed by contacting the host cell with a LacI repressor protein allosteric effector. The LacI allosteric effector may cause a conformational shift in a LacI repressor protein. The conformational shift may decrease the affinity of LacI repressor protein for a lac operator. The LacI allosteric effector may lead to expression of the gene of interest. The LacI allosteric effector may be lactose. The LacI allosteric effector may also be a lactose analog, which may be isopropyl-β-D-thiogalactopyranoside (IPTG).

### 5. METHOD OF CLONING

Also provided herein is a method for cloning a first nucleic acid. The method may involve using RecA protein in a the host cell to induce homologous recombination between the first nucleic acid and a second nucleic acid.

a. First Nucleic Acid

The first nucleic acid may be a circular nucleic acid. The first nucleic acid may be the product of a polymerase chain reaction followed by circularization, such as by intramolecular DNA ligation. The first nucleic acid may also be located on a vector. The first nucleic may comprise a selectable marker that may be capable of indicating whether the first nucleic acid is present in the host cell. The selectable marker may comprise a gene capable of conveying antibiotic resistance, such as to kanamycin, and may also comprise a visible marker such as green fluorescent protein.

b. Second Nucleic Acid

The second nucleic acid may be capable of being replicated in the host cell. The second nucleic acid may be a vector such as a plasmid or a host cell chromosome. The second nucleic acid may comprise a selectable marker. The second nucleic acid may also comprise at least one sequence that is identical or substantially identical to the first nucleic acid.

c. RecA

The RecA protein may be capable of inducing homologous recombination in the host cell. The RecA may comprise a sequence as set forth in SEQ ID NO: 20, or a variant thereof that is capable of inducing homologous recombination. The RecA may be encoded by a recA gene, which may be located on a vector, such as a plasmid or a host cell chromosome. The recA gene-containing vector may comprise a selectable marker. The recA gene may be expressed from a constitutive or regulatable promoter. The gene may be isolated from a bacterial strain such as E. coli, and may comprise a sequence as set forth in SEQ ID NO: 21.

d. Homologous Recombination

The first and second nucleic acids may be recombined by contacting them under conditions that favor homologous recombination. The first and second nucleic acids may be introduced into the host cell, such as by transformation. The host cell may already comprise the second nucleic acid, and the host cell may be transformed with the first nucleic acid. The host cell may be selected for the presence of the selectable marker of the first nucleic acid.

The host cell may comprise the recA gene. The host cell may also be transformed with a nucleic acid comprising the recA gene. The recA gene may be induced to express recA protein.

The first and second nucleic acids may be contacted in the host cell, in which the recA protein may induce homologous recombination between the first and second nucleic acids. Expression of the recA may then be stopped, such as by removing the inducer of the recA gene expression. Removing the selection agent for the recA gene-containing plasmid and allowing the host cell to lose the plasmid may also stop recA expression. A host cell comprising a product of homologous recombination between the first and second nucleic acids may be selected by selecting for the presence of the selectable marker contained by the first nucleic acid.

The present invention has multiple aspects, illustrated by the following non-limiting examples.

### Example 1

### Making the lac Super Operator T7 Polymerase Construct

This example describes the construction of a lac super operator T7 polymerase nucleic acid for use in homologous recombination into the E. coli genome. The polymerase chain reaction (PCR) was used to amplify four sections of DNA in a first round of PCR, as follows.

PCR1: a 3′ portion of the lacI gene of E. coli strain MG1655 was amplified using the primer pair 5′-TGGGTCACCAGCAATCGCGCTG (primer o-mc-1, SEQ ID NO: 22) and 5′-CATAGCTGTTTCCTGTGGAATTGTGAGCGCTCACAATTCCACACAACATAC (o-mc-2, SEQ ID NO: 23). PCR2: T7 RNA Polymerase was amplified from bacteriophage T7 DNA using the primer pair 5′-GCTCACAATTCCACAGGAAACAGCTATGAACACGATTAACATCGCT AAGAAC (o-mc-3, SEQ ID NO: 24) and 5′-CAGACATGGCCTGCCCGGTTATTATTATT TTTGACACCAGACCATTACGCGAACGCGAAGTCCGAC (03-lac T7-Egg 3, SEQ ID NO: 25).

PCR3: The kanamycin resistance gene was amplified from strain FB21288 (available from the University of Wisconsin E. coli Genome Project, Madison, Wis.) using the primer pair 5′-ACCAATTACCCTGTTATCCCTATTAGAAAAACTCATCGAGCATCAAATG (03-Km-lac L, SEQ ID NO: 28) and 5′-AATAACCGGGCAGGCCATGTCTGCCCGTAGGGATAACA GGGTAATCAGTAATACAAGGGGTGTTATGAG (03-lac-Egg Km 3, SEQ ID NO: 29).

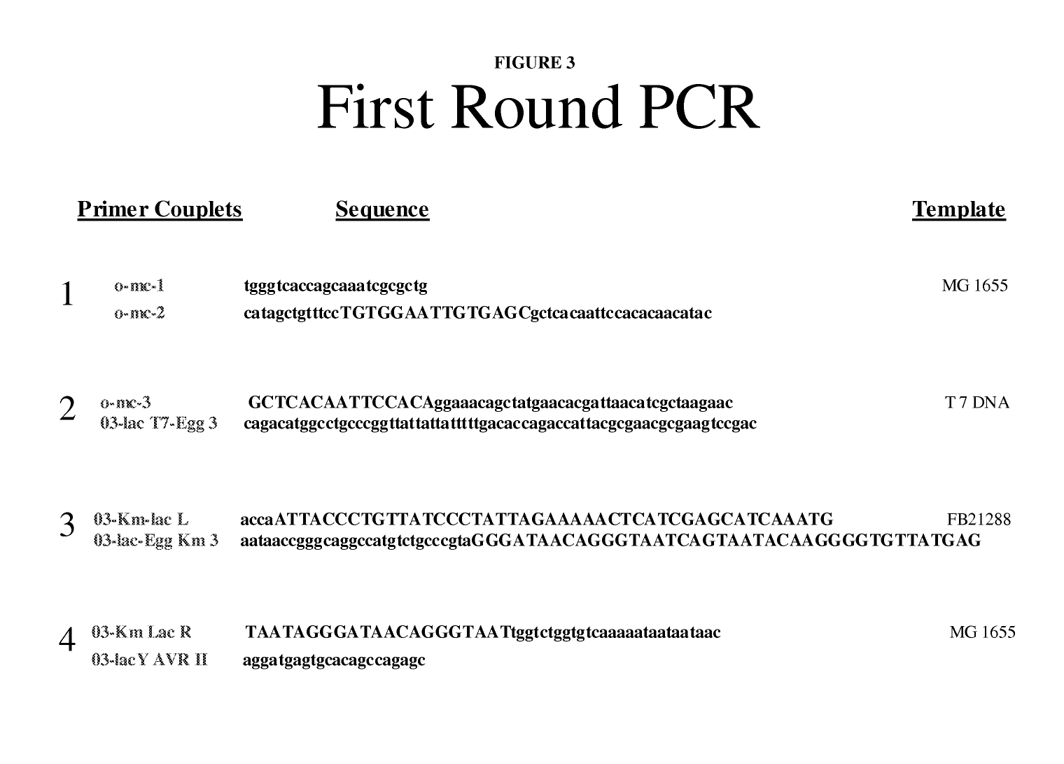

PCR4: A 5′ portion of the E. coli lacY gene was amplified from E. coli MG1655 using the primer pair 5′-TAATAGGGATAACAGGGTAATTGGTCTGGTGTCAAAAATAATAATAAC (03-Km Lac R, SEQ ID NO: 26) and 5′-AGGATGAGTGCACAGCCAGAGC (03-lac Y AVR II, SEQ ID NO: 27). The primers and templates from the first round of PCR are shown in FIG. 3.

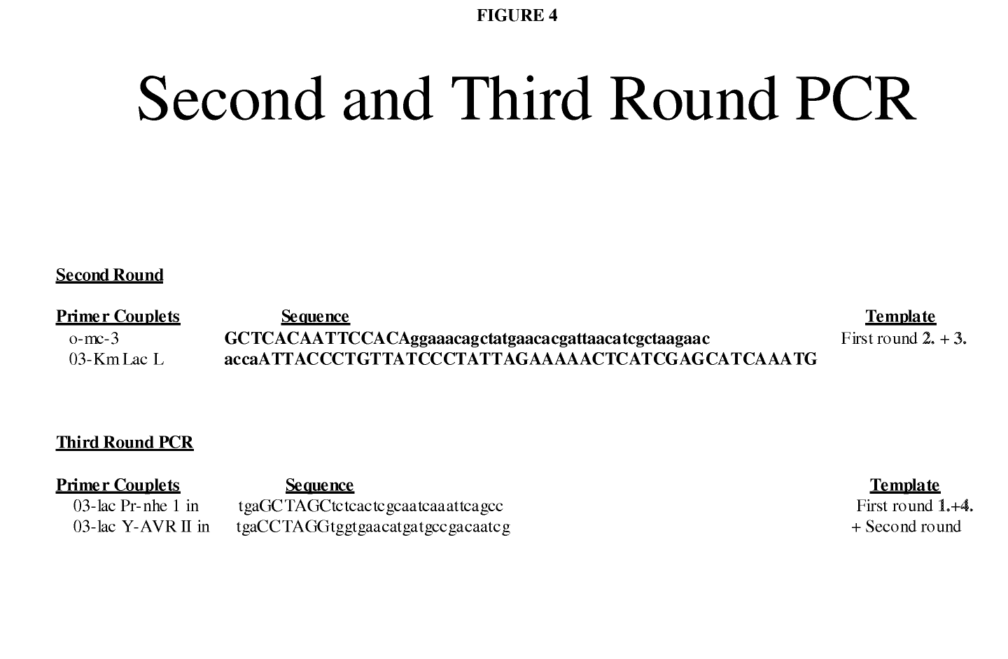

After purifying the products of the PCR reactions above, a second round of PCR was performed by using the primers o-mc-3 and 03-Km Lac L to amplify a combination of the products of PCR2 and PCR3 above. After purifying the product of the second round of PCR, a third round of PCR was performed by using the nested primer pair 5′-TGAGCTAGCTCTCACT CGCAATCAAATTCAGCC (03-lac Pr-nhe 1 in, SEQ ID NO: 30) and 5′-TGACCTAGGT GGTGAACATGATGCCGACAATCG (03-lac Y-AVR II in, SEQ ID NO: 31) to amplify the product of the second round of PCR together with the products of PCR1 and PCR4 from the first round of PCR. The primers and templates from the second and third round of PCR are shown in FIG. 4.

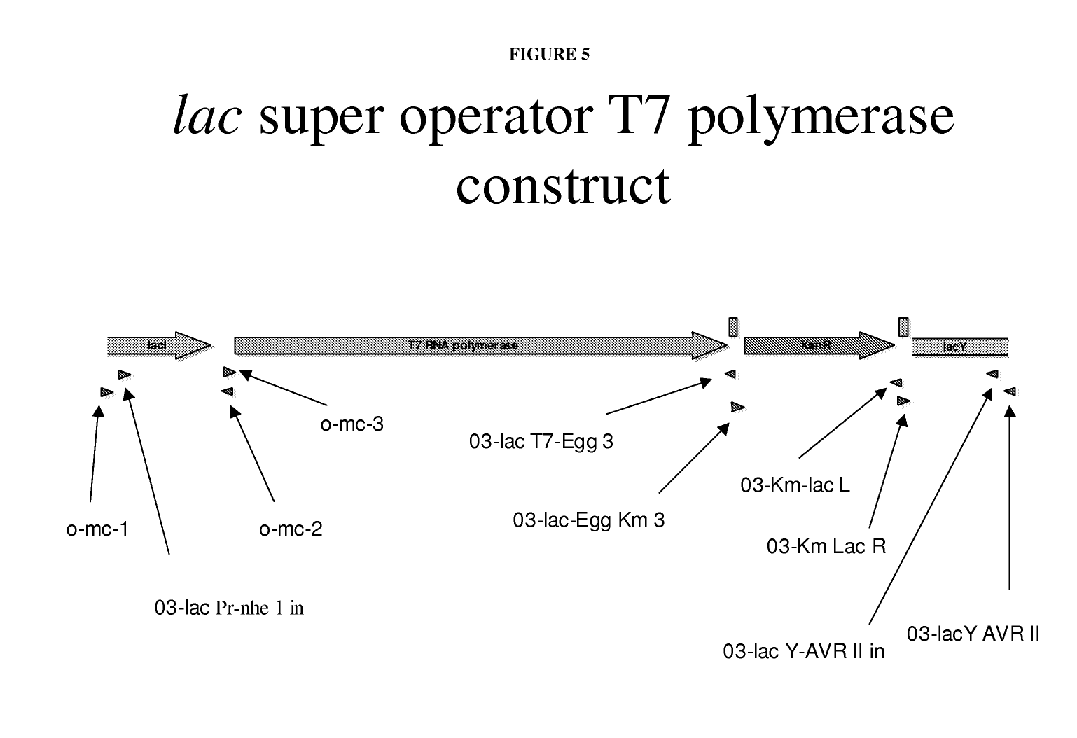

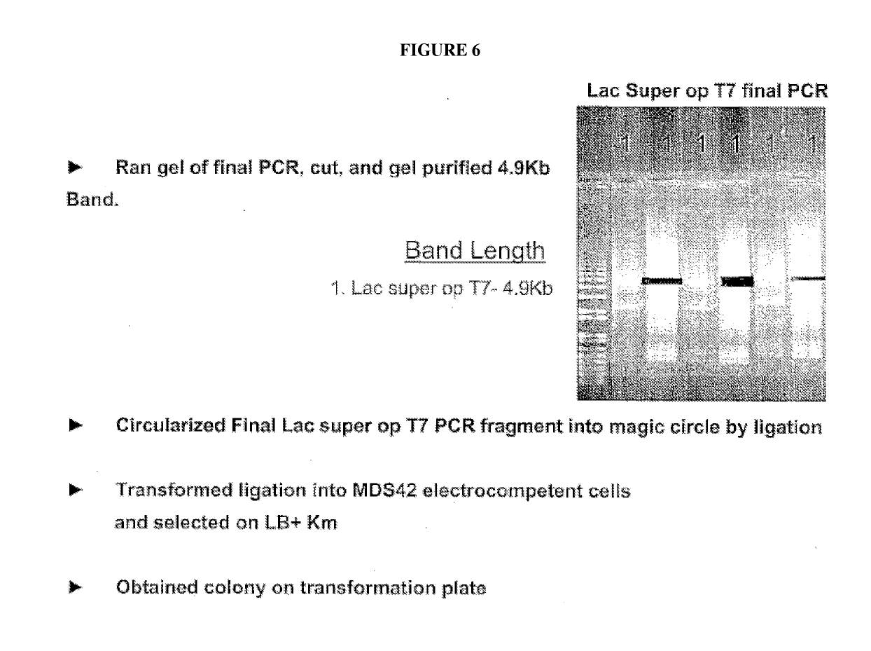

A schematic of the final lac-T7 polymerase construct and the primers used to make it are shown in FIG. 5. The primers o-mc-2 and o-mc-3 in PCR1 and PCR2 introduced a mutant lac operator sequence (5′-GCTCACAATTCCACA; SEQ ID NO: 32) upstream of T7 polymerase. The mutant operator has enhanced affinity for LacI. The lac “super operator” T7 polymerase construct thus comprised the following components, from 5′ to 3′: (#1) a 3′ portion of E. coli lacI, (#2) T7 RNA polymerase with a lac super operator, (#3) a short 5′ portion of E. coli lacY, (#4) the kanamycin resistance gene and (#5) a 5′ portion of E. coli lacY. The final 4.9 kb PCR product was gel purified. A schematic of the gel purification and a photograph of the gel is shown are FIG. 6.

### Example 2

### Recombination of the lac Super Operator T7 Polymerase Construct into the E. coli Chromosome

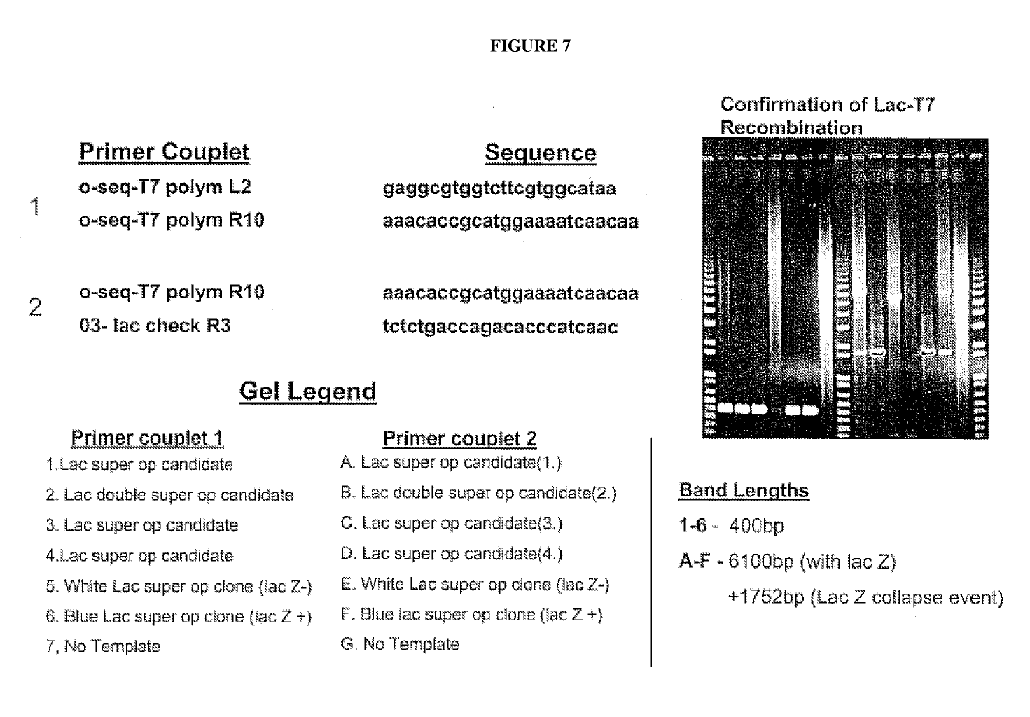

This example describes the results of recombining the lac super operator T7 polymerase construct into E. coli. The lac super operator T7 polymerase construct from Example 1 was circularized via ligation. Electrocompetent MDS42 E. coli cells were transformed with the circularized construct and recombinants between the circularized construct and the E. coli chromosome were selected for on a LB+Km plate. A schematic of this strategy is shown in FIG. 6. Candidate recombinants (kanamycin-resistant blue colonies on X-Gal plates) were further screened via PCR using primer couplet 1, comprising primers with the sequences 5′-GAGGCGTGGTCTTCGTGGCATAA (o-seq-T7 polym L2, SEQ ID NO: 33) and 5′-AAACACCGCATGGAAAATCAACAA (o-seq-T7 polym R10, SEQ ID NO: 34), and primer couplet 2, comprising o-seq-T7 polym R10 and a primer with the sequence 5′-TCTCTGACCAGACACCCATCAAC (03-lac check R3, SEQ ID NO: 35). A schematic and the results of the PCR screen are shown in FIG. 7.

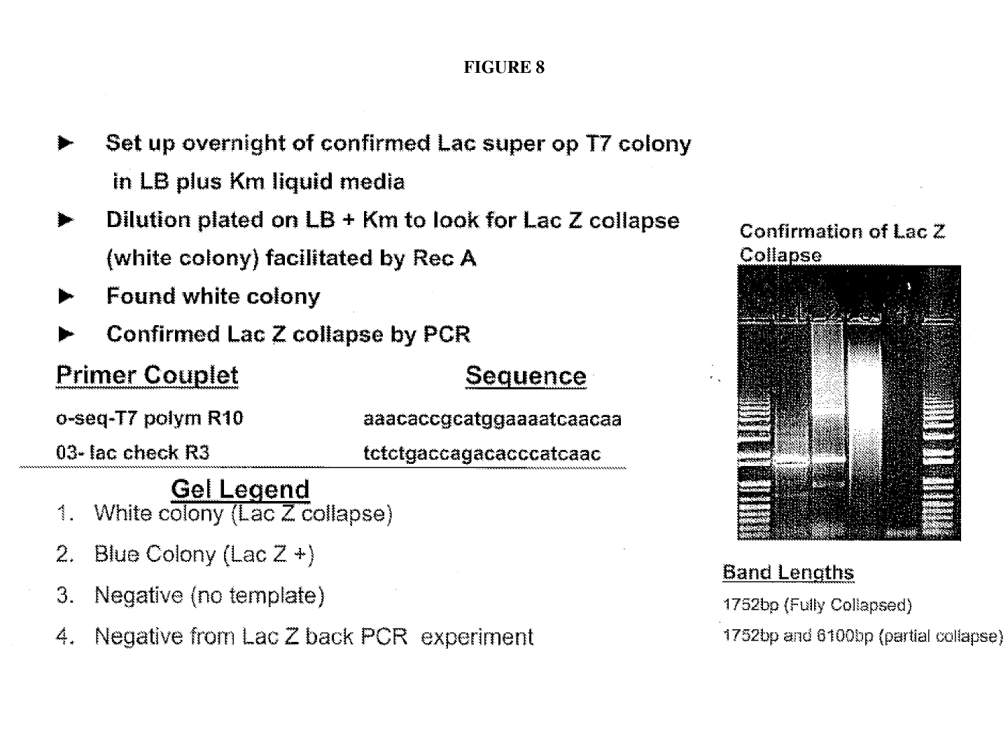

Next, colonies that had produced a ˜6.1 kb PCR fragment with the o-seq-T7 polym R10 and 03-lac check R3 primers were cultured overnight in LB plus Km liquid media. Dilutions were plated on LB+Km+X-Gal and IPTG to screen for colonies that were white. White colonies were predicted to have undergone an intramolecular RecA-mediated recombination event between the 3′ portion of lacI (#1 in Example 1) and the E. coli chromosomal copy of lacI, which resulted in “collapse” of the region of the E. coli chromosome in which the endogenous lacZ resided. Colonies of clones in which lacZ collapsed were screened via PCR using primers o-seq-T7 polym R10 and 03-lac check R3. Amplification reactions that yielded a 1.752 kb PCR products confirmed a lacZ collapse. A schematic and the results of the screen are shown in FIG. 8.

### Example 3

### Construction of a lacZ Back Construct

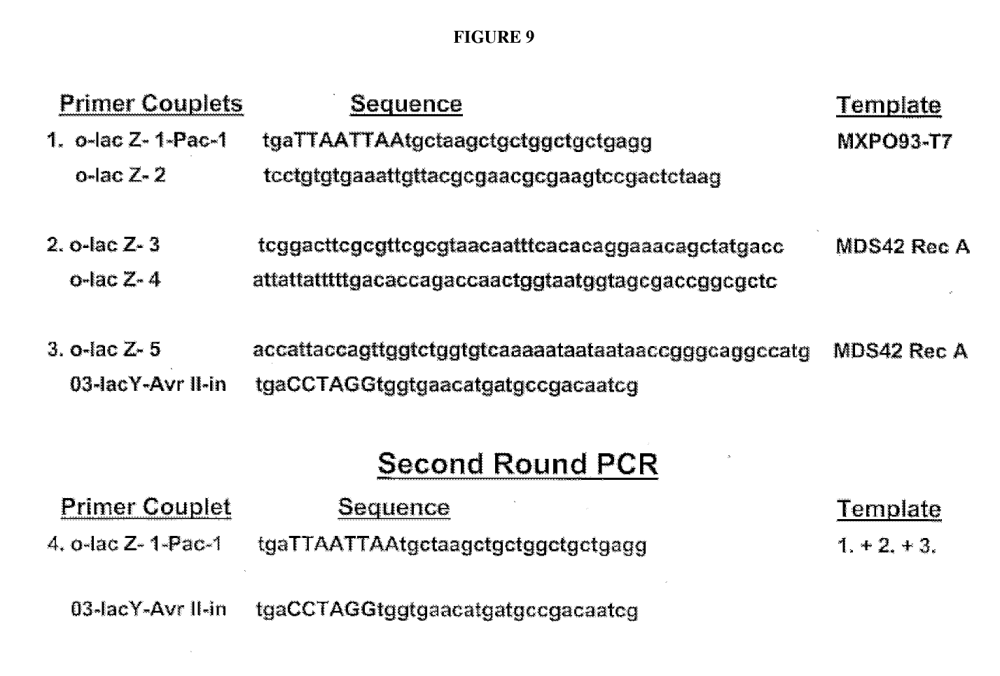

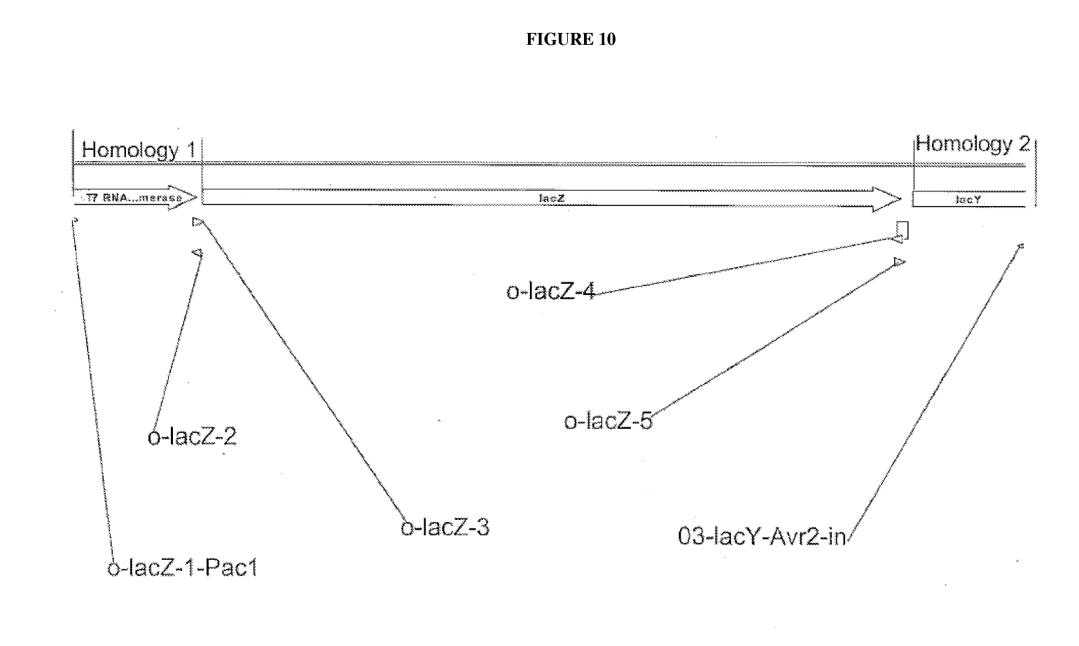

This example describes generation of a T7 RNA polymerase lacZ back construct. PCR was used to amplify three DNA fragments, as follows. PCRa: A short segment of the 3′ end of T7 gene 1 was PCR amplified from bacteriophage T7 DNA using the primers 5′-TGATTAATTAATGCTAAGCTGCTGGCTGCTGAGG (o-lac Z-1-Pac-1, SEQ ID NO: 36) and 5′-TCCTGTGTGAAATTGTTACGCGAACGCGAAGTCCGACTCTAAG (o-lac Z-2, SEQ ID NO: 37). PCRb: The lacZ gene was PCR amplified from E. coli strain MDS42recA genomic DNA using the primers 5′-TCGGACTTCGCGTTCGCGTAACAATTTCACACAGG AAACAGCTATGACC (o-lac Z-3, SEQ ID NO: 38) and 5′-ATTATTATTTTTGACACCAG ACCAACTGGTGAATGGTAGCGACCGGCGCTCk (o-lac Z-4, SEQ ID NO: 39). PCRc: A short segment of the 5′ end of the lacY gene was PCR amplified from E. coli strain MDS42recA genomic DNA using the primer 5′-ACCATTACCAGTTGGTCTGGTGTCAAAAATA ATAATAACCGGGCAGGCCATG (o-lac Z-5, SEQ ID NO: 40) and the primer 03-lac Y-AVR II from Example 1. After isolating the three PCR products, they were combined and amplified using the primers o-lac Z-1-Pac-1 and 03-lacY-Avr II in. The primers and templates used are shown in FIG. 9. A schematic of the lacZ back construct and the primers used to construct it are shown in FIG. 10. The lacZ back construct comprised, as follows, from 5′ to 3′: (1) a 3′ portion of T7 RNA polymerase, (2) lacZ, and (3) a 5′ portion of lacY.

### Example 4

### Introduction of lacZ into a lac Super Operator T7 Polymerase E. coli Strain

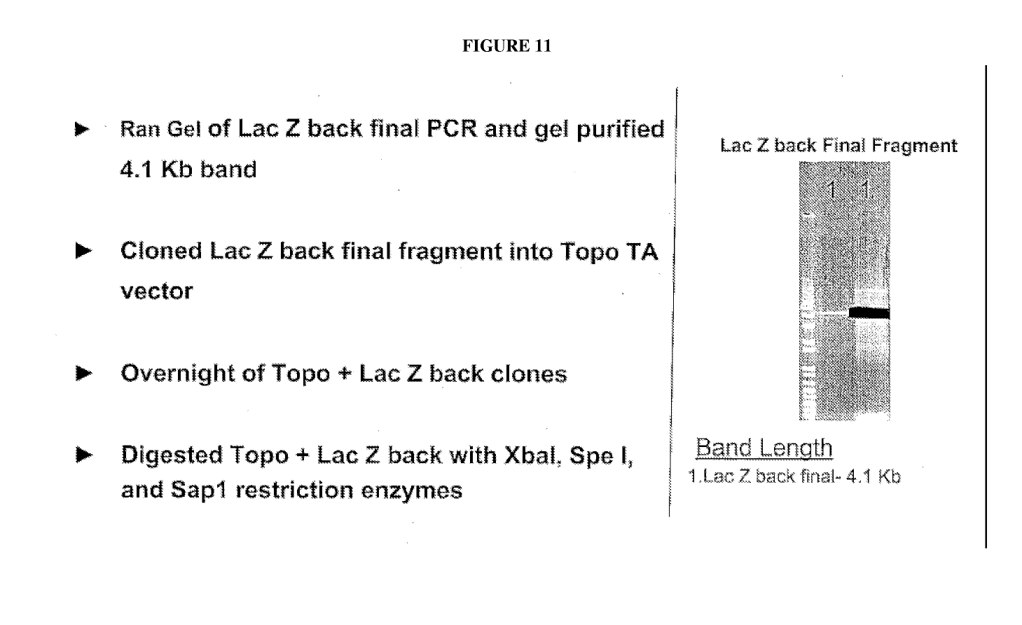

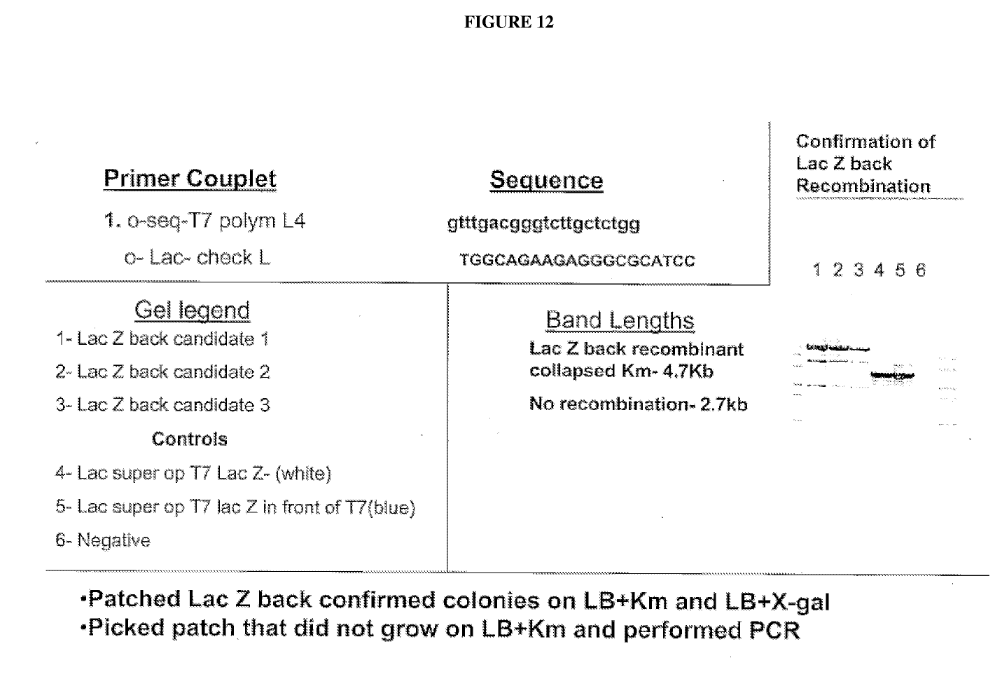

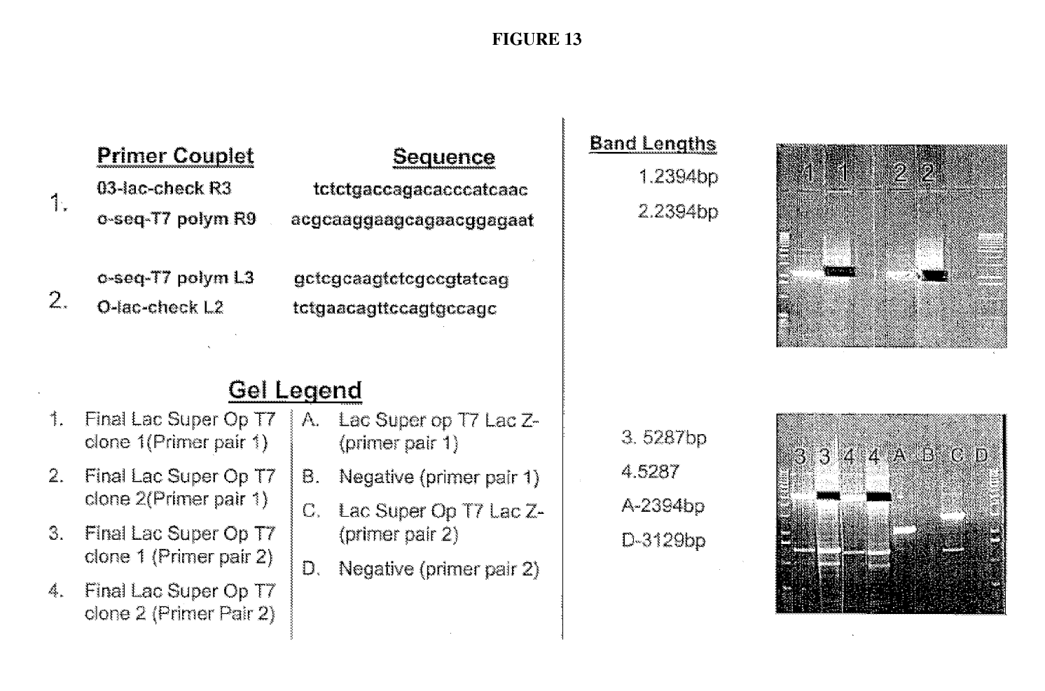

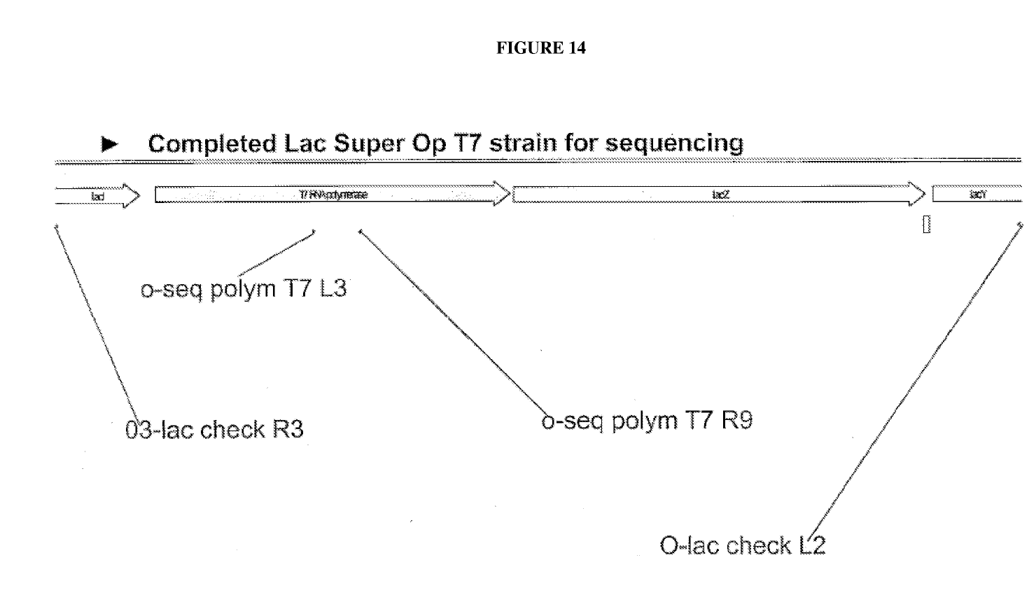

This example describes generating a lac super operator T7 E. coli strain capable of catabolizing lactose. The 4.1 kb lacZ back product of Example 3 was gel purified and cloned into a Topo TA vector by incubating the lacZ back product and Topo TA vector with DNA topoisomerase I overnight. The Topo+lacZ back plasmid clones were prepared and digested with XbaI, SpeI, and SapI. The cloning strategy and a photograph of the gel are shown in FIG. 11. Next, the lacZ back fragment was circularized by ligation. The ligation products were transformed into electrocompetent lac super operator T7/lacZ collapsed cells from Example 2. Clones were selected on MMM+0.2% lactose+X-Gal. Blue colonies were picked and confirmed for successful lacZ back recombination via PCR by using the primers 5′-GTTTGACGGGTCTTGCTCTGG (o-seq-T7 polym L4, SEQ ID NO: 41) and 5′-TGGCAGAAGAGGGCGCATCC (o-Lac-check L, SEQ ID NO: 42). The PCR screening strategy and results are shown in FIG. 12. Clones yielding a ˜2.7 kb PCR fragment were patched on LB+Km and LB+X-Gal. Colonies that did not grow on LB+Km were selected and confirmed by PCR using primer pair LacZ1: 5′-TCTCTGACCAGACACCCATCAAC (03-lac-check R3, SEQ ID NO: 43) and 5′-ACGCAAGGAAGCAGAACGGAGAAT (o-seq-T7 polym R9, SEQ ID NO: 44), and LacZ2: 5′-GCTCGCAAGTCTCGCCGTATCAG (o-seq-T7 polym L3, SEQ ID NO: 45) and 5′-TCTGAACAGTTCCAGTGCCAGC(O-lac-check L2, SEQ ID NO: 46). Colonies that yielded a ˜2.4 kb fragment with primer pair LacZ1 and a ˜5.3 kb fragment with primer pair LacZ2 were confirmed to contain a lac super operator T7 polymerase and lacZ, and lack kanamycin resistance, were selected for sequencing. The primers and the PCR results are shown in FIG. 13. A schematic of the final lac super operator T7 strain and the primers use to construct it are shown in FIG. 14.

1. A nucleic acid comprising a mutant lac operator operably linked to a gene of interest, wherein the mutant lac operator has increased affinity for a LacI repressor protein.

2. The nucleic acid of claim 1, wherein the nucleic acid is a plasmid.

3. The nucleic acid of claim 1, wherein the nucleic acid is a chromosome.

4. The nucleic acid of claim 1, wherein the increased affinity is at least 5-fold.

5. The nucleic acid of claim 4, wherein the sequence of the mutant lac operator comprises any one of SEQ ID NOS: 2-6.

6. The nucleic acid of claim 1, wherein the gene of interest is T7 gene 1.

7. The nucleic acid of claim 1, wherein the gene of interest is trfA.

8. A host cell comprising the nucleic acid of claim 1.

9. The host cell of claim 8 further comprising a lacI gene.

10. The host cell of claim 9, wherein the lacI gene is a mutant lacI allele.

11. The host cell of claim 10, wherein the mutant lacI allele encodes a mutant LacI repressor protein that is capable of binding a lac operator with increased affinity.

12. The host cell of claim 8 further comprising an inactive lacZ gene.

13. A method of expressing a gene of interest comprising:
(a) providing the host cell of claim 8;
(b) contacting the host cell with a LacI allosteric effector,
wherein the LacI allosteric effector leads to expression of the gene of interest.

14. The method of claim 13 wherein the LacI allosteric effector is derived from lactose.

15. The method of claim 13 wherein the LacI allosteric effector is a lactose analog.

16. The method of claim 15 wherein the lactose analog is isopropyl-β-D-thiogalactopyranoside.

17. A method of cloning a first nucleic acid, comprising:
(a) providing a host cell, wherein the host cell comprises recA;
(b) providing a first nucleic acid, wherein the first nucleic acid is circular;
(c) providing a second nucleic acid; and
(d) contacting the first nucleic acid with the second nucleic in the host cell, wherein first nucleic acid recombines with the second nucleic acid.

18. The method of claim 17, wherein the recA is located in the host cell genome.

19. The method of claim 17, wherein the recA is located on a plasmid.

20. The method of claim 19, wherein the recA plasmid is removed after the first nucleic acid and second nucleic acid recombine.

21. The method of claim 17, wherein the first nucleic acid comprises at least two regions of sequence identity to regions on the second nucleic acid.

22. The method of claim 17, wherein the second nucleic acid is a host cell chromosome.

23. The method of claim 17, wherein the first nucleic acid is a plasmid.

24. The method of claim 23, wherein the plasmid comprises a selectable marker.

25. The method of claim 24, wherein the selectable marker conveys kanamycin resistance.

26. The method of claim 25, wherein the selectable marker further green fluorescent protein.

27. The method of claim 17, wherein the host cell is a gram-negative bacterium.

28. The method of claim 27, wherein the bacterium is E. coli.
